(19) United States
(12) Patent Application Publication
(10) Pub. No.: US 2010/0016322 A1
(43) Pub. Date: Jan. 21, 2010

(54) Water Dispersible Pharmaceutical Formulation and Process for Preparing The Same

(76) Inventors: Nagesh Nagaraju, Karnataka (IN); Prakash Kumar Soni, Madhya Pradesh (IN); Gour Mukherji, Haryana (IN)

Correspondence Address: COHEN, PONTANI, LIEBERMAN & PAVANE LLP, 551 FIFTH AVENUE, SUITE 1210, NEW YORK, NY, 10176, US

(21) Appl. No.: 12/528,859

(22) PCT Filed: Feb. 27, 2008
(86) PCT No.: PCT/IN2008/000111

§ 371 Date: Aug. 27, 2009

(30) Foreign Application Priority Data
Feb. 28, 2007 (IN) 444/DEL/2007

(51) Int. Cl. A61K 31/53

(52) U.S. Cl. 514/242

(57) ABSTRACT

Water dispersible compressed tablets and a process for preparing the same. The tablet comprising about 0.1 to 50% w/w of lamotrigine or its pharmaceutically acceptable salts, solvates, hydrates or polymorphs, about 5 to about 50% w/w of a water-soluble diluent(s), about 15 to about 70% w/w of a water swellable diluent(s), optionally one or more pharmaceutically acceptable adjuvants, wherein the ratio of water-soluble diluent(s) to water swellable diluents(s) is from about 0.6 to about 0.9 and said composition is essentially free of disintegrant, superdisintegrant and swellable clay.

### PRIORITY CLAIM

This is a U.S. national stage of application No. PCT/IN2008/000111, filed on Feb. 27, 2008. Priority is claimed on the following application(s): Country: India, Application No.: 444/DEL/2007, Filed: Feb. 28, 2007, the content of which is/are incorporated here by reference.

### FIELD OF THE INVENTION

The present invention relates to a water dispersible compressed tablet of a pharmaceutically active ingredient or its pharmaceutically acceptable salts, solvates, hydrates or polymorphs for oral administration. The invention also provides a process for manufacturing said tablet.

### BACKGROUND OF THE INVENTION

Therapeutically active ingredients are frequently administered to patients in the form of a tablet or capsule, when the active ingredient is intended for oral administration. Tablets and capsules are convenient pharmaceutical dosage forms for manufacture, storage and ensure dosage uniformity. However, such dosage forms, like capsules and tablets, often present ingestion problems such as difficulty in swallowing for the debilitated patients, particularly for pediatric and geriatric populations. This may result in a high incidence of non-compliance and ineffective therapy, which may prove to be fatal in case of serious conditions.

Suspension dosage forms could solve this problem, but they have other associated drawbacks like lower physical and chemical stability and high cost of manufacturing. Suspensions are also inconvenient to carry while travelling and also involve the risk of inaccurate measurement and dosing.

Thus, there is a need for oral pharmaceutical composition, which can be taken orally without the need of swallowing it and act as a viable substitute for suspensions. Accordingly, provided are water dispersible tablet compositions, which can either be chewed or can be readily dispersed in water before oral administration

One of the key requirements of water dispersible tablet is that they should dissolve in an aqueous medium within a short time period of, for example, less than three minutes, to form a smooth suspension without any coarse lumps.

Well known methods of producing dispersible tablets, generally involved the incorporation of high amount of disintegrating agents in the tablets. Disintegrants are the essential ingredients in the dispersible tablets as they impart desirable properties like rapid swelling or good wetting to the tablets. Additionally, in most of the cases, best disintegration is achieved by the use of combination of two or more disintegrating agents.

U.S. Pat. No. 4,886,669, assigned to Zyna SA, discloses a pharmaceutical composition of one or more pharmacologically active ingredients in the form of water-dispersible tablet comprising of active ingredient in the form of microparticles. The formulation also contains at least one disintegrant and a swellable material, which generates high viscosity when it comes in contact with water. The microparticles are coated and have a specific size range and are prepared for controlled release or taste masking purposes. The disintegrant disclosed is crospovidone.

U.S. Pat. No. 5,556,639, assigned to Glaxo, discloses the use of disintegrating agents (incorporated both internally and externally to the preformed granules) such as sodium starch glycolate, cross-linked povidone and cross-linked sodium carboxymethylcellulose, along with swellable clay to produce a satisfactory water dispersible tablet. Furthermore, an ion exchange resin (Amberlite IRP88) and surface-active agents were also employed as a co-disintegrating agent and incorporated in order to improve tablet wetting and penetration of water during dispersion formation, resulting in low disintegration time. The said patent thus utilizes an unique property of a known adjuvant-Veegum®, a swellable clay, during granulation which is essential to effect proper dispersion.

U.S. Pat. No. 5,698,226, assigned to the Wellcome Foundation Inc, discloses water dispersible tablets of lamotrigine. These tablets are made up of lamotrigine, pharmaceutically acceptable swellable clay, additional pharmaceutically acceptable secondary disintegrating agents and other excipients, wherein the tablet thus prepared is capable of dispersing in water within a period of three minutes. The patent further states that the secondary disintegrating agents alone do not provide desirable disintegrating properties to the tablet. Accordingly, the applicant states that it is advantageous to use swellable clay as an adjunct for the dispersion to provide adequate disintegration of the tablets in water. However, the use of swellable clays can undesirably retard the disintegration times of the tablet. Moreover, the use of swellable clays in the manufacture of tablets is avoided due to their off-white appearance, which causes discoloration in tablets. Also, clays are prone to microbial contamination due to their origin from natural sources.

U.S. Pat. No. 5,861,179, assigned to the Wellcome Foundation Inc, discloses a powder formulation of lamotrigine that includes lactose, starch, crystalline cellulose and polyvinyl pyrrolidone, wherein, combination of starch and crystalline cellulose serves the role of disintegrant.

U.S. Pat. No. 5,047,247, assigned to Lek, discloses the dispersible tablet of dihydroxergotoxine methanesulfonate prepared on the basis of known method of granulating the ingredients. However, it needs to be noted that granulation step is time consuming. The avoidance of this step may lead to shortening of time cycle for a batch production and, reduction in manpower, utilities and equipment costs associated with the said step of granulation. The said patent further discloses that disintegrants are essential elements of the dispersible tablets that imparts desirable properties like rapid swelling or good wetting to the tablets.

US Application No. 20030186938, assigned to SmithKline, refers to a water-dispersible formulation for immediate oral administration that comprises a dry blend of paroxetine, a water-soluble dispersing agent selected from crosslinked polyvinylpyrrolidone, calcium carbonate and sodium starch glycolate, and a taste-masking agent. The said formulation is presented either as a dispersible powder or moulded into a tablet.

US Application No. 20050238724, assigned to Teva Pharmaceutical Industries, discloses a tablet formulation containing lamotrigine, diluents, binders and disintegrants.

International Publication No. WO2004103340, assigned to Ranbaxy Laboratories, discloses a water-dispersible tablet comprising granules or compacts. Said granules or compacts comprising lamotrigine or its pharmaceutically acceptable salts and one or more disintegrants. The disintegrants include cross-linked polyvinylpyrrolidone, sodium starch glycolate, cross-linked sodium carboxymethyl cellulose, calcium carboxymethyl cellulose, alginic acid and alginates, pregelatinized starch, starch and starch derivatives, low-substituted hydroxypropyl cellulose or combinations thereof.

International Publication No. WO2004006917, assigned to Ranbaxy Laboratories, relates to a process for the preparation of a dispersible tablet dosage form comprising beta-lactam antibiotics for oral administration and a disintegrating agent being used both intragranularly and extragranularly.

International Publication No. WO2004014337, assigned to Ranbaxy Laboratories, relates to dispersible tablets of cephalexin comprising granulation of cephalexin along with a disintegrating agent and colloidal silicon dioxide with binder solution to form granules, drying and mixing said granules with disintegrating agents, fillers, lubricants and optionally, other excipients.

International Publication No WO2005051350, assigned to Torrent Pharmaceuticals, discloses water dispersible tablets of lamotrigine with the use of disintegrant but without the use of swellable clay, wherein primary disintegrant includes cross-linked derivatives of cellulose, crospovidone and starch or modified starches.

International Publication No WO2007013047, assigned to Ranbaxy Laboratories, discloses water dispersible tablets of antiretroviral drugs. In the application, it is mentioned that the disintegrants play a major role in the disintegration of water dispersible tablets. Disintegrants used are sodium starch glycolate, cross-linked carboxymethylcellulose and its sodium salt, cross-linked polyvinylpyrrolidone, pregelatinized starch, sodium carboxymethyl cellulose, calcium carboxymethyl cellulose, low-substituted hydroxypropyl cellulose and alginates.

Furthermore, all the prior arts cited above teach that the use of disintegrant is essential for the formulation of any water dispersible tablet. Normally dispersible tablets have high percentages of disintegrating agents. Moreover, the presence of such ingredients tends to weaken the tablet's structure, leading to exacerbated friability and low hardness values. Nonetheless, there is considerable room for improvement.

An objective of the present invention is to avoid the above-mentioned inconvenience and to provide a water dispersible compressed tablet comprising a pharmaceutically active ingredient with fast dispersion which will pass swiftly through a 710 μm diameter mesh size sieve (dispersion quality), which is capable of dispersing in water within 3 minutes, preferably within 2 minutes and most preferably within 1 minute, having an enhanced structural integrity, for instance having a friability lower than 1.0%, with a pleasant taste and the absence of perceptible granules in the mouth. The present invention provides a simple and easy process of manufacturing of such a composition.

Dispersible tablets are usually prepared using a superdisintegrant along with a co-disintegrant to provide satisfactory disintegration and dissolution time. In stark contrast, the inventors of the present invention have surprisingly discovered that a water dispersible compressed tablet of low solubility active ingredient can be formulated by interplaying with a combination of water-soluble diluents and water swellable diluents. Preparation of water dispersible tablets of the present invention, which are essentially free of any disintegrant or superdisintegrant or swellable clay and with enhanced organoleptic characteristics, is a highly challenging task for a person skilled in the art.

The present invention includes oral water dispersible pharmaceutical compositions in the form of a compressed tablet of pharmaceutically active ingredient or its pharmaceutically acceptable salts with a desired disintegration and/or dispersion time wherein said tablet is essentially free of disintegrant or superdisintegrant or swellable clay.

### SUMMARY OF THE INVENTION

The principal aspect of the present invention encompasses a stable water dispersible tablet composition comprising a pharmaceutically active ingredient or its pharmaceutically acceptable salts and the process of manufacturing such a composition.

In one aspect of the present invention, there is provided a water dispersible pharmaceutical composition in the form of compressed tablets for oral administration comprising an effective amount of a pharmaceutically active ingredient or its pharmaceutically acceptable salts, solvates, hydrates or polymorphs, water soluble diluent(s), water swellable diluent(s) and optionally one or more pharmaceutically acceptable adjuvants wherein said pharmaceutically acceptable adjuvant is not a disintegrant nor superdisinegrant nor swellable clay and yet said composition is having a desired disintegration and/or dispersion time with a good mouthfeel. The water dispersible tablet of the present invention is capable of dispersing in water within a period of three minutes (dispersion time) to provide a dispersion which is capable of passing through a sieve screen with a mesh aperture of 710 μm (dispersion quality) in accordance with the test for dispersible tablets as defined in British Pharmacopoeia 1988, volume II, page 895, wherein said tablet is a stable water dispersible compressed tablet.

In accordance with another aspect of the present invention, there is provided a water dispersible compressed tablet comprising an effective amount of a pharmaceutically active ingredient or its pharmaceutically acceptable salts, water soluble diluent(s), water swellable diluent(s) and optionally one or more pharmaceutically acceptable adjuvants wherein said pharmaceutically acceptable adjuvants are selected from binders, lubricants, glidants, surfactants, sweeteners, flavors, stabilizers or any possible combination thereof.

In accordance with another aspect of the present invention, there is provided a water dispersible compressed tablet, wherein the ratio of water soluble diluent(s) to water swellable diluent(s) varies preferably from about 0.6 to about 0.9.

In accordance with another aspect, the tablet of the present invention is essentially free of disintegrant, superdisintegrant and swellable clay.

In accordance with another aspect, the present invention describes the process for manufacturing a water dispersible compressed tablet of pharmaceutically active ingredient or its pharmaceutically acceptable salts having dispersion time of less than three minutes, more preferably less than 2 minutes and most preferably less than 1 minute. The composition of the present invention can be manufactured either by wet granulation or dry granulation or direct compression method.

In accordance with a further aspect of the present invention, provided are processes for manufacturing a water dispersible compressed tablet comprising the steps: a) forming a blend comprising a pharmaceutically active ingredient or its pharmaceutically acceptable salts, water soluble diluent(s), a water swellable diluent(s) and optionally one or more pharmaceutically acceptable adjuvants selected from binders, lubricants, glidants, surfactants, sweeteners, flavors, stabilizers or any combination thereof; b) finally compressing the blend into a tablet.

In accordance with yet another aspect of the present invention, there is provided a process for manufacturing a water dispersible compressed tablet comprising the steps: a) forming a blend comprising pharmaceutically active ingredient(s) or its pharmaceutically acceptable salts, water swellable diluent(s), optionally water soluble diluent(s) and optionally one or more pharmaceutically acceptable adjuvants selected from binders, lubricants, glidants, surfactants, stabilizers, flavors, sweeteners or any combination thereof; b) granulating the blend by wet or dry granulation to form granules; c) blending the granules with water soluble diluent(s), optionally water swellable diluent(s) and optionally one or more pharmaceutically acceptable adjuvants selected from surfactants, lubricants, glidants, sweeteners, flavors, stabilizers or any combination thereof and d) finally compressing the blend into a suitable size and shape tablet.

In accordance with further another aspect, the present invention provides a method of treating various disorders or diseases employing said pharmaceutically active ingredient or its pharmaceutically acceptable salts in the form of a water dispersible compressed tablet as described. The method comprises administering said tablet comprising a pharmaceutically active ingredient or its pharmaceutically acceptable salts thereof, water soluble diluent(s), water swellable diluent(s) and optionally one or more pharmaceutically acceptable adjuvants, wherein said tablet is essentially free of disintegrant or superdisintegrant or swellable clay.

The water dispersible compressed tablets of the present invention are stable as demonstrated by accelerated stability testing as per ICH guidelines.

The details of one or more embodiments of the inventions are set forth in the description below. Others features, objects and advantages of the inventions will be apparent from the description and claims.

### DETAILED DESCRIPTION OF THE PRESENTLY PREFERRED EMBODIMENTS

The invention will now be described specifically in terms of its most preferred embodiments including stable water dispersible compressed tablet composition comprising a pharmaceutically active ingredient or its pharmaceutically acceptable salts. Reference will also be made in detail herein to other preferred embodiments of the compositions, processes and methods of the invention.

The present invention provides a stable pharmaceutical composition of a pharmaceutically active ingredient or its pharmaceutically acceptable salts for oral administration in the form of a water dispersible compressed tablet and the process for manufacturing such pharmaceutical composition.

The present invention provides a process for the manufacturing of a stable water dispersible compressed tablet of a pharmaceutically active ingredient or its pharmaceutically acceptable salts which, when dispersed in water gives a non-gritty homogeneous dispersion in less than about three minutes, more preferably less than 2 minutes and most preferably less than 1 minute. The water dispersible tablet is capable of dispersing in water within a period of three minutes to provide dispersion, which is capable of passing through a sieve screen with a mesh aperture of 710 μm in accordance with the test for dispersible tablets defined in British Pharmacopoeia 1988, volume II, page 895.

Dispersible tablets in accordance with the present invention are intended to be dispersed in water prior to administration, resulting in a homogenous dispersion. However, the dispersible tablets can generally also be chewed and/or swallowed.

According to the present invention, the term ‘water dispersible compressed tablet’ is used synonymously with the term “water dispersible tablet”.

According to the present invention, the term “composition” is used synonymously with the term “formulation”.

In general, the water dispersible compressed tablet in accordance with the present invention can accommodate a wide variety of pharmaceutical active ingredients. The pharmaceutically active ingredient (s) are present in a therapeutically effective amount, which is an amount that produces the desired therapeutic response upon oral administration and can be readily determined by one skilled in the art. In determining such amounts, the particular pharmaceutically active ingredient being administered, the bioavailability characteristics of the pharmaceutically active ingredient, the dose regime, the age and weight of the patient, and other factors must be considered, as known in the art.

The amount of active ingredient used can vary greatly. The amount of active ingredient to be incorporated in the tablet of the present invention largely depends on the dose requirement of the active ingredient. Generally, the active ingredient is provided in an amount of about 0.01 to about 50% by weight of the tablet, more preferably, from about 0.05 to about 40% by weight and still more preferably in an amount of about 0.05 to about 35% by weight of the tablet thereof.

A non exhaustive listing of suitable pharmaceutical actives from which the pharmaceutically active ingredient may be chosen include the class of anti-epileptic agents, anti-histaminic, anti-viral agents, anti-asthmatics, neuroleptics, anti-migraine agents, anxiety agents, general anesthetics, anti-acne, anti-diarrheal agents, anti-infectives, antibiotics, anti-microbials, laxatives, anti-convulsants, anti-anginals, diuretics, transquilizers, anti-depressants, anti-spasmodics, anti-allergics, bronchodilators, hypnotics, sedatives, gastrointestinal agents, anti-hypertensives (diuretics, beta-blockers, ACE inhibitors, calcium channel blockers, vasodilators), hypotensives, vasoconstrictors, hypoglycemic agents, antipyretics-analgesics-anti-inflammatory agents, anti-arrhythmic agents, chemotherapeutics, anti-bacterials, anorexics, antiflatulents, anti-fungal, anti-protozoals, cyclooxygenase inhibitor, cough suppressants, mucolytics, anti-uricemic agents, central nervous system acting agents, β-lactam antibiotics, psychotropic agents, sympathomimetics, anti-emetic agents, anti-diabetic agents, decarboxylate inhibitor, antirheumatics, hormone drugs, antacids, decongestants, anti-coagulants, anti-platelet agents, skeletal muscle relaxants, anti-thyroid, hypoglycemic agents, neuroprotective agents, neuromuscular agents, immunomodulating agents, serotonin receptor agonist, serotonin receptor antagonist, serotonin uptake inhibitor, drugs for osteoporosis, drugs for Parkinson's disease, nourishing and health promoting agents such as vitamins and minerals, and the like.

In particular, pharmaceutically active ingredient may include, but not limited to, montelukast, lamotrigine, zolmitriptan, rizatriptan, sumatriptan, naratriptan, olanzapine, risperdone, cetirizine, ranitidine, diclofenac, domperidone, amoxicillin, ondansetron, granisetron, piroxicam, nabumetone, ibuprofen, naproxen, flurbiprofen, diclofenac, acyclovir, deferacorix, acetaminophen, desloratadine, fexofenadine, levodopa, carbidopa, delavirdine, doxycycline, cefixime, oxcarbazepine, famotidine, metformin, glipizide, clarithromycin, azithromycin, cyclandelate, mirtazapine, 5-HT reuptake inhibitor such as fluoxetine, paroxetine, citalopram, escitalopram, sertraline, benzimidazole derivatives such as lansoprazole, timoprazole, pantoprazole, leminoprazole, rabeprazole; steroids such as prednisolone, prednisone, hydrocortisone or their pharmaceutically acceptable salts, solvates, hydrates or polymorphs.

In addition, it is possible to use any salts and free base form of pharmaceutically active ingredient including polymorphs, hydrates and solvates as well as amorphous forms of active ingredient. As used in the present specification and claims “pharmaceutically active ingredient” will be deemed to encompass the free base and pharmaceutically acceptable salts, polymorphs, hydrates and solvates as well as amorphous forms of active ingredient.

The phrase “stable pharmaceutical composition” herein refers to a composition of pharmaceutically active ingredient in which there is no substantial change in assay values, impurity percentages, hardness, dispersion quality and dissolution values when kept at 40° C.±2° C./75% RH±5% RH for 3 months.

By “dispersible tablet” is meant a tablet, which disperses in aqueous phase, for example, in water before administration. A water-dispersible tablet, according to the British Pharmacopoeia and European Pharmacopoeia, should meet the requirements of the test for dispersible tablets as regards dispersion time (<3 minutes) and dispersion quality (i.e. to pass through a 710 μm sieve).

The term “homogeneous dispersion” as used herein means that the dispersion is produced upon contact with water, which ensures the uniformity of the pharmacologically active ingredient content for a reasonable period of time.

Unless otherwise specified, the term “about” has the meaning given to it by those well acquainted with the arts to which this invention pertains and where not in conflict with the understanding of skilled artisans, by its customary and accepted meaning.

Provided herein are stable water dispersible pharmaceutical compositions for oral administration in the form of a compressed tablet comprising a pharmaceutically active ingredient or its pharmaceutically acceptable salts, water soluble diluent(s), water swellable diluent(s) and optionally one or more pharmaceutically acceptable adjuvants selected from binders, lubricants, glidants, surfactants, sweeteners, flavors, stabilizers or any combination thereof, wherein said tablet is essentially free of disintegrant or superdisintegrant or swellable clay and yet having a desired disintegration and/or dispersion time with a good mouthfeel.

While the exact composition of these water dispersible compressed tablets can vary, it has been observed that it is advantageous to formulate them in a way so that ratio of water-soluble diluent(s) to water swellable diluent(s) can vary from about 0.3 to about 1.5, preferably from about 0.5 to about 1.2, more preferably from about 0.6 to about 0.9.

One embodiment of the present invention encompasses a water dispersible compressed tablet comprising about 0.01% to about 50% w/w of the pharmaceutically active ingredient or its pharmaceutically acceptable salts, about 5% to about 50% water soluble diluent(s), about 35% to about 70% w/w of water swellable diluent(s) and optionally one or more pharmaceutically acceptable adjuvants, wherein the ratio of water soluble diluent(s) to water swellable diluent(s) is from about 0.6 to about 0.9 and said tablet is essentially free of disintegrant or superdisintegrant or swellable clay.

Another embodiment of the present invention encompasses a water dispersible compressed tablet comprising about 0.01% to about 50% w/w of the pharmaceutically active ingredient or its pharmaceutically acceptable salts, about 5% to about 50% water soluble diluent(s), about 35% to about 70% w/w of water swellable diluent(s) and optionally one or more pharmaceutically acceptable adjuvants, wherein the ratio of water soluble diluent(s) to water swellable diluent(s) is from about 0.6 to about 0.9 and said tablet is essentially free of disintegrant, superdisintegrant and swellable clay.

In another example, a water dispersible compressed tablet can comprise about 0.01% to 50% w/w of lamotrigine or its pharmaceutically acceptable salts, solvates, hydrates or polymorphs, about 5% to about 50% w/w of a water-soluble diluent(s), about 35% to about 70% w/w of a water swellable diluent(s), optionally one or more pharmaceutically acceptable adjuvants, wherein the ratio of water-soluble diluent(s) to water swellable diluent(s) is from about 0.6 to about 0.9 and said tablet is essentially free of disintegrant or superdisintegrant or swellable clay.

In another example, a water dispersible compressed tablet can comprise about 0.01% to 50% w/w of montelukast or its pharmaceutically acceptable salts, solvates, hydrates or polymorphs, about 5% to about 50% w/w of a water-soluble diluent(s), about 35% to about 70% w/w of a water swellable diluent(s), optionally one or more pharmaceutically acceptable adjuvants, wherein the ratio of water-soluble diluent(s) to water swellable diluent(s) is from about 0.6 to about 0.9 and said tablet is essentially free of disintegrant or superdisintegrant or swellable clay.

In another example, a water dispersible compressed tablet can comprise about 0.01% to 50% w/w of olanzapine or its pharmaceutically acceptable salts, solvates, hydrates or polymorphs, about 5% to about 50% w/w of a water-soluble diluent(s), about 35% to about 70% w/w of a water swellable diluent(s), optionally one or more pharmaceutically acceptable adjuvants, wherein the ratio of water-soluble diluent(s) to water swellable diluent(s) is from about 0.6 to about 0.9 and said tablet is essentially free of disintegrant or superdisintegrant or swellable clay.

In one embodiment, a water dispersible compressed tablet can comprise about 0.01% to 50% w/w of olanzapine or its pharmaceutically acceptable salts, solvates, hydrates or polymorphs, about 5% to about 50% w/w of a water-soluble diluent (s), about 35% to about 70% w/w of a water swellable diluent (s), optionally one or more pharmaceutically acceptable adjuvants, wherein the ratio of water-soluble diluent(s) to water swellable diluent(s) is from about 0.6 to about 0.9 and said tablet is essentially free of disintegrant or superdisintegrant.

In one embodiment the tablet of the present invention is essentially free of swellable clay.

Another embodiment of the present invention encompasses a water dispersible compressed tablet comprising pharmaceutically active ingredient or its pharmaceutically acceptable salts, water soluble diluent(s), water swellable diluent(s) and optionally one or more pharmaceutically acceptable adjuvants, wherein said tablet is essentially free of superdisintegrant like crospovidone, croscarmellose, sodium starch glycolate and low substituted hydroxypropylcellulose and free of swellable clay like bentonite, magnesium and other aluminium silicate such as Veegum® and the like.

The “adjuvants” used in accordance with the present invention include pharmaceutically acceptable compounds which are intended to enhance the handling and/or manufacturing of the pharmaceutical composition into an acceptably uniform, flowable and compressible admixture which can be readily produced into the final dosage form. By “pharmaceutically acceptable” is meant a material which is not biologically or otherwise undesirable, i.e., the material can be administered to an individual along with the active ingredient without causing any undesirable biological effects or interacting in a deleterious manner with any of the components of the composition in which it is contained.

The water dispersible compressed tablet in accordance with the present invention include water-soluble diluents and water swellable diluents. Diluents and standard pharmaceutically acceptable adjuvants such as binders, surfactants, lubricants, glidants, sweeteners, flavors, stabilizers, coloring agents and the like can be used to make up the bulk of the tablet composition and to provide the requisite flowability, moldability, compatibility, integrity and palatability. These adjuvants have been elaborated herein. Those skilled in the art will appreciate that the amount of adjuvants may vary depending on the strength of particular adjuvants used and the level approved by regulatory authorities for use in pharmaceutical products.

Unlimited examples of water-soluble diluents in accordance with the present invention include, for example, lactose, mannitol, xylitol, sorbitol, calcium sulfate dihydrate, inositol, dextrin, calcium sulfate anhydrous, fructose, kaolin, sugar compressible, sucrose, lactitol, dextrates, confectioner's sugar, sucrose, sodium chloride, dextrose or any combination thereof and the like. In particular, granulated (known as mannitol granules) or spray-dried mannitol (commercially available under the brand name Pearlitol®SD 200 from Roquette, France) is found to be useful, especially in compositions intended for direct compression process. Mannitol and other similar compounds having a negative heat of solution are preferred because they provide a particularly pleasant sensation enhancing organoleptic experience of taking the tablet of the present invention. According to the present invention, the amount of water-soluble diluents may vary within a range of from about 5% to about 50%, preferably from about 10% to about 40%, still more preferably from about 30% to about 40% by weight of the tablet.

Water swellable diluents include, but are not limited to, pregelatinized starch, starch, microcrystalline cellulose, powdered cellulose, silicified microcrystalline cellulose, dibasic calcium phosphate dihydrate, calcium phosphate, calcium carbonate, hydroxypropylcellulose, hydroxyethylcellulose, hydroxypropyl methylcellulose, polyethyleneglycol, xanthum gum, gum arabic or any combination thereof and the like. Microcrystalline cellulose is commonly used as a diluent in tablets and commercially available under the trade name Emcocel™ (supplied by Edward Mendell of New York) and Avicel™ (supplied by FMC Corporation of Philadelphia, Pa.). Particular grades of Avicel™ include Avicel PH 101, 102, 103, 105 and 200. In addition, microcrystalline cellulose imparts an almost creamy mouth feel, which helps to offset the negative impact of its swellability. The water swellable diluents may be present in the tablet in an amount of from about 35% to about 70% by weight of the tablet, more particularly from about 40% to about 70%, preferably from about 45% to about 65%, still more preferably from about 50% to about 60% by weight of the tablet.

The ratio of water-soluble diluent(s) to water swellable diluent(s) varies from about 0.3 to about 1.5, preferably from about 0.5 to about 1.2, still more preferably from about 0.6 to about 0.9, such ratio is critical for the present invention.

Binders are generally used in a solid dosage form to facilitate the compression of the powdery material and give the tablet strength. Suitable binders include, but are not limited to, polyvinylpyrrolidone, starch mucilage, pregelatinized starch, dextrin, guar gum, maltose, sodium alginate, alginic acid, acacia mucilage, tragacanth, hydroxypropyl methylcellulose, carboxymethylcellulose sodium, carboxymethylcellulose calcium, ethyl cellulose, polyethylene glycol, hydroxyethyl cellulose, hydroxypropyl cellulose, methyl cellulose, polymethacrylates (like, Eudragit®), carboxyvinyl polymers (like, Carbopol®), polyvinyl alcohol and other such materials routinely used in the art of solid dosage form manufacturing. Binders, may be a dry binder, such as, microcrystalline cellulose, which can be particularly useful in a direct compression and dry granulation process. For the composition of the present invention, the preferred binder is polyvinylpyrrolidone. Polyvinylpyrrolidone is commercially available from BASF, Germany, under the brand name of Kollidon®K30. The binder may be present in an amount of about 1% to about 40%, particularly about 1% to about 30%, and more particularly about 1% to about 20% by weight of the tablet.

Lubricants and glidants are adjuvants used in the formula to reduce inter-particle and die-wall friction and enhance the powder flow due to their large surface area. Suitable examples of lubricants include, but are not limited to, talc, magnesium stearate, calcium stearate, zinc stearate, sodium lauryl sulphate, sodium stearyl fumarate (Pruv), glyceryl behenate, stearic acid, polyethylene glycol, glyceryl palmitostearate and monostearate and the like. The preferred lubricant is magnesium stearate. Magnesium stearate is commercially available from Mallinkrodt. The lubricant may be present from about 0.1% to about 5% by weight of the tablet, particularly about 0.5 to about 4% and more particularly about 1% to about 3% by weight of the tablet.

Suitable glidants of the present invention include, but are not limited to, talc, colloidal silicon dioxide, amorphous silicon dioxide, magnesium silicate and calcium silicate. The preferred glidant is colloidal silicon dioxide. Colloidal silicon dioxide is commercially available from Degussa, Germany, under the brand name, Aerosil®. Similarly, amorphous silicon dioxide is commercially available as RxCIPIENTS® and Syloid®244 FP from J.M.Huber Corporation, USA and Grace GmbH, Germany, respectively. Preferably glidants are used in an amount of about 0.1 to about 5% and more preferably about 0.5 to about 3% by weight of the tablet.

For the purpose of the invention, colloidal silicon dioxide also includes colloidal silica or its derivatives such as Syloid®. Colloidal silicon dioxide serves two purposes, first as glidants and then as a suspension aid. A particular amount of colloidal silicon dioxide can be from about 0.25 to about 5.0% by weight of the tablet.

Surfactants are added to improve wetting of the tablets. Appropriate surfactants according to the invention include, but are not limited to, sodium lauryl sulfate, quaternary ammonium salts, polysorbates, sorbitan esters and/or poloxamer. Preferably, the surfactant is sodium lauryl sulfate. The amount of surfactant may vary from about 0 to about 3%, preferably from about 0.01 to 1.5% by weight of the tablet.

For an active ingredient prone to degradation, one or more stabilizers may also be included in the composition. Stabilizers include compounds such as any antioxidation agents, pH modulators etc. Antioxidants that may be used include but are not limited to, tocopherols and derivatives thereof, ascorbic acid, sodium ascorbate, calcium ascorbate, ascorbic palmitate, beta-carotene, selenium, sodium bisulfite, tocopherol, tocopherol acetate, tocopherol acid succinate, propyl gallate, vitamin E, sodium metabisulfite, ascorbyl palmitate, butylated hydroxyanisol (BHA), butylated hydroxytoluene (BHT) and mixtures thereof. pH modifiers or buffers may also be used to maintain the pH of the final composition within a certain desired range.

It is within the scope of the invention to include various other conventional ingredients known in the art for improving organoleptic characters of the pharmaceutical composition such as sweeteners and flavors.

Sweeteners are used to improve the taste of the composition. Sweeteners used in the present invention may be artificial sweeteners such as, for example, aspartame, sodium saccharin, cyclamate, acesulfame potassium, maltodextrin, dextrates, sucralose natural sugars or other pharmacologically acceptable sweeteners known from the state of the art. Artificial sweeteners are preferred in the present invention because of the interest of the formulation to be administered to diabetics. Sweeteners are used in an amount of from about 0.01 to about 5% by weight of the tablet. In the preferred embodiment the sweetener is aspartame (Nutrasweet), which is used in an amount ranging from about 0.5 to about 3% by weight of the tablet.

Water dispersible compressed tablet of the present invention may contain any flavoring agent approved by FDA for oral use so long as they do not materially affect the physical or chemical attributes of the active or of the resulting suspension. Both natural and synthetic flavorants are contemplated for use herein. In the present invention flavoring agents may preferably be selected among conventional flavors, such as black current flavor, strawberry flavor, raspberry flavor, banana flavor, mint flavor, lemon flavor, peach flavor, grape fruit flavor, orange flavor, mixed fruit flavor, menthol, citric acid, numeric acid, peppermint flavor or any possible combination thereof. Preferably, flavoring agents are used in an amount of from about 0.5 to about 5% by weight of the tablet. Those skilled in the art of compounding pharmaceutical medicaments will appreciate that the amount of flavors and sweeteners, if any, present in the composition will be directly proportional to the bitterness which has to be masked.

Coloring agents may also be incorporated in the pharmaceutical composition to provide an appealing color to the composition. The coloring agents should be selected to avoid chemical incompatibilities with other ingredients in the composition. Suitable coloring agents are known to those skilled in the art.

Optionally other pharmaceutically acceptable adjuvants may be selected and used having regard to the particular desired properties of the dispersible tablet by routine experimentation.

For active ingredients having a bitter taste, taste-modifying components are employed in an effective amount to produce a consumer acceptable suspension upon disintegration of the dispersible tablet. The amount of taste modifying components required would vary with the amount of pharmaceutical active ingredient used as well as the intensity of the poor taste of the pharmaceutical active ingredient.

Taste modifying compositions in accordance with the invention include but are not limited to sugars, sweet polyhydric alcohols, glycerin, artificial sweetener, flavoring agents and mixtures thereof. Examples of sugars include sucrose, fructose, dextrose, and glucose. Examples of sweet polyhydric alcohols include sorbitol and mannitol. Examples of high intensity sweeteners include aspartame, sucralose, cyclamates, acesulfame potassium, saccharin and mixtures thereof.

Another aspect of the present invention is the discovery of an interesting synergy, which exists by the use of water soluble diluents in combination with water swellable diluents in the water dispersible compressed tablet described herein, which leads to reduced disintegration and/or dispersion time aiding in uniform dispersion generation to enhance the overall performance of these compositions.

“Disintegration time” is the time for the tablet to disintegrate in water at room temperature in a disintegration time device.

In one of the embodiments, the water dispersible compressed tablet of the present invention may be manufactured by conventional processes known to those of ordinary skill in the art, including, but not limited to, direct compression, wet granulation or dry granulation such as slugging or compaction of the composition into tablet. All these methods are well known in the art, and are described in detail, for example, Lachman et al., “The theory and Practice of Industrial Pharmacy” which is incorporated herein by reference.

Typically, water dispersible compressed tablets are manufactured using wet granulation method as it results in the formation of softer, more porous granules, which can disintegrate in aqueous solution to give a smooth suspension, avoiding the presence of coarse lumps. We have found that the water dispersible tablet of the present invention can also be prepared by dry granulation or direct compression to yield tablets which gives smooth suspension upon disintegration. The pharmaceutical composition of the present invention can be prepared by a straightforward procedure. The process for preparation of the dispersible tablets of this invention is very simple, cost effective, production not requiring any special equipment and does not require any hazardous solvent.

In another preferred embodiment, a stable water dispersible compressed tablet of a pharmaceutically active ingredient is prepared by direct compression. The process includes a) forming a blend comprising a pharmaceutically active ingredient or its pharmaceutically acceptable salts, water soluble diluent(s), water swellable diluent(s) and optionally one or more pharmaceutically acceptable adjuvants selected from one or more binders, lubricants, glidants, surfactants, stabilizers, flavors, sweeteners or any combination thereof; and b) finally compressing the blend into a tablet.

In yet another embodiment, there is provided a process for preparing a water dispersible compressed tablet for oral administration comprising the steps of: a) forming a blend comprising a pharmaceutically active ingredient or its pharmaceutically acceptable salts, water swellable diluent(s), optionally water soluble diluent(s) and one or more pharmaceutically acceptable adjuvants selected from binders, lubricants, glidants, surfactants, stabilizers, flavors, sweeteners or any combination thereof; b) granulating the blend with a solution or dispersion of one or more binders to form granules; c) alternatively, one or more binders may be added to the above blend and the resulting blend granulated with one or more suitable solvents; d) drying the resultant granules, and if needed, sized; e) blending the granules with water soluble diluent(s), optionally water swellable diluent(s) and one or more pharmaceutically acceptable adjuvants selected from lubricants, glidants, surfactants, stabilizers, sweeteners, flavors; and f) finally compressing the blend into a tablet.

In further embodiment, the composition of the present invention may be prepared by slugging or roller compaction. Particularly suitable is roller compaction. In some embodiments, a therapeutically effective amount of a pharmaceutically active ingredient, water soluble diluents, water swellable diluents and one or more of pharmaceutical adjuvants selected from binders, sweeteners, glidants, lubricants or any combination thereof are blended and transferred to a roller compactor and compacted into a sheet. The resulting compact sheet may be fed to a mill, such as an oscillatory mill filled with a screen. After passing through the mill and the screen, the compact can be converted into granules of desired particle size distribution. The granules may further be mixed with water soluble diluent(s), water swellable diluent(s) and one or more pharmaceutically acceptable adjuvants selected from binders, lubricants, glidants, surfactants, sweeteners, flavors or any combination thereof and resulting blend then compressed into a tablet.

Thus, the present invention provides a process of manufacturing a water dispersible compressed tablet comprising a therapeutically effective amount of a pharmaceutically active ingredient together with an effective amount of a pharmaceutically acceptable water soluble diluent and a water swellable diluent which comprises combining the active ingredient with said diluents to provide a water dispersible tablet which is capable of dispersing in water within a period of three minutes to provide a dispersion which is capable of passing through a sieve screen with a mesh aperture of 710 μm in accordance with the test for dispersible tablets as defined in the British Pharmacopoeia, 1988, Volume II, page 895.

Various equipments used for the manufacturing of the composition of the present invention include, for example, for blending, simple blending equipments known in the art can be used. For granulation, mixer granulator or fluid bed processor or any other convenient granulating equipment can be used. For dry granulation, roller compactor or any similar equipment can be used. Drying of granules can be carried out in fluid bed processor itself or other drying techniques can be employed such as tray drying, vacuum drying or flash drying. Mixing of dried granules with lubricants can be carried out in a suitable mixer like double cone or V blender. The tabletting of lubricated granules can be carried out in a suitable tabletting machine equipped with suitable tooling.

Therefore, the water dispersible compressed tablet of the present invention can be used for the therapy or prophylaxis of various diseases just as the conventional preparations containing the same active ingredient but with an increased ease of ingestion by elderly persons and children.

Most preferably, the water dispersible tablets of the present invention are contained in packaging materials, which protect the tablets from moisture and light. For example, suitable packaging material includes amber colored high-density polyethylene bottles, amber colored glass bottles and other container made of a material, which inhibits passage of light.

The hardness of the tablet of the present invention remains substantially constant and leads to constant dispersion time. The tablets prepared by these processes or operations have sufficient hardness and less friability to withstand handling and storage. Typically, the tablet has a friability of 2% or less, particularly 1% or less, and more particularly 0.5% or less. The tablet disperses quickly in water to give a fine dispersion, which is free from chalkiness and provides the intended dose uniformly. The tablets can also be swallowed as such like conventional tablets. The tablet disperses completely in water in about 2 minutes, preferably in about 1 minute.

The invention will be better understood from the following examples. However, one skilled in the art will readily appreciate the specific material and results described are merely illustration of, and are not intended to, nor should be intended to, limit the invention as described more fully in the claims which follow thereafter. Accordingly, it is not intended that the scope of the claims appended hereto and following examples to be interpreted to the exact description set forth, but rather that the claims be construed as encompassing all of the features of patentable novelty that reside in the present invention, including all the features and embodiments that would be treated as equivalent thereof by those skilled in the art to which the invention pertains. Unless otherwise stated, the percentages given in the description and examples are by weight.

### Example 1

TABLE 1
Lamotrigine water dispersible tablet

| S. No | Ingredients | Quantity (mg/tab) | % w/w |
|---|---|---|---|
| 1. | Lamotrigine | 25.0 | 8.33 |
| 2. | Spray dried mannitol | 100.0 | 33.33 |
| 3. | Microcrystalline cellulose | 163.0 | 54.33 |
| 4. | Aspartame | 6.0 | 2.00 |
| 5. | Colloidal silicon dioxide | 2.0 | 0.70 |
| 6. | Magnesium stearate | 3.0 | 1.00 |
| 7. | Strawberry flavor | 1.0 | 0.33 |

| | | |
|---|---|---|
| Total | 300.0 | 100 |

### Manufacturing Procedure:

1) Accurately weighed quantities of lamotrigine, spray dried mannitol, microcrystalline cellulose, aspartame, colloidal silicon dioxide and strawberry flavor were mixed together in a geometric order in a double cone blender.
2) The blend of step 1 was sifted through ASTM #40 mesh and uniformly mixed in a double cone blender for 10 minutes.
3) The weighed quantity of magnesium stearate was sifted through ASTM #40 mesh and added to the blend of step 2 and uniformly mixed in a double cone blender for 3 minutes.
4) The blend of step 3 was compressed using flat-faced beveled edge punches to form compressed tablets.

TABLE 2
Physicochemical properties of the tablet

| Parameter | Observed value |
|---|---|
| Hardness | 7-9 kp |
| Friability | 0.11% |
| Disintegration time | 19-21 sec |
| Dispersion quality test | No particles retained on 710 μm sieve |

### Example 2

TABLE 3
Lamotrigine water dispersible tablet

| | | | |
|---|---|---|---|
| S. No | Ingredients | Quantity (mg/tab) | % w/w |

Intragranular

| | | | |
|---|---|---|---|
| 1. | Lamotrigine | 25.0 | 5.00 |
| 2. | Microcrystalline cellulose | 100.0 | 20.00 |
| 3. | Aspartame | 3.0 | 0.60 |
| 4. | Polyvinyl pyrrolidone K-30 | 10.0 | 2.00 |
| 5. | Purified Water* | Q.S | |

Extragranular

| | | | |
|---|---|---|---|
| 6. | Microcrystalline cellulose | 180.0 | 36.00 |
| 7. | Spray dried mannitol | 170.5 | 34.10 |
| 8. | Strawberry flavor | 0.5 | 0.10 |
| 9. | Aspartame | 4.0 | 0.80 |
| 10. | Magnesium stearate | 4.0 | 0.80 |
| 11. | Colloidal silicon dioxide | 3.0 | 0.60 |

| | | |
|---|---|---|
| Total | 500.0 | 100 |

*Evaporates during processing

### Manufacturing Procedure:

1) Lamotrigine, microcrystalline cellulose and aspartame were sifted through ASTM #40 mesh and uniformly mixed in a double cone blender.
2) The accurately weighed quantity of polyvinyl pyrrolidone was dissolved in purified water using magnetic stirrer.
3) The blend of step 1 was granulated with solution of step 2 in rapid mixer granulator for 5 minutes.
4) The wet mass of step 3 was sifted through ASTM #25 mesh.
5) The granulates of step 4 were transferred to a tray drier and dried at 60° C. for 15 minutes.
6) The weighed quantities of microcrystalline cellulose, spray dried mannitol, colloidal silicon dioxide and strawberry flavor were sifted through ASTM #40 mesh.
7) The weighed quantity of granulates of step 5 were added to the blend of step 6 and uniformly mixed in a double cone blender for 10 minutes.
8) The weighed quantity of magnesium stearate was added to the blend of step 7 and uniformly mixed in a double cone blender for 3 minutes.
9) The resultant granulates were compressed using flat-faced beveled edge punches to form compressed tablets.

### Example-3

TABLE 4
Lamotrigine water dispersible tablet

| S. No | Ingredients | Quantity (mg/tab) | % w/w |
|---|---|---|---|
| 1. | Lamotrigine | 25.0 | 8.33 |
| 2. | Spray dried mannitol | 103.0 | 34.33 |
| 3. | Microcrystalline cellulose | 163.0 | 54.33 |
| 4. | Aspartame | 2.0 | 0.70 |
| 5. | Acesulfame potassium | 1.0 | 0.33 |
| 6. | Colloidal silicon dioxide | 2.0 | 0.70 |
| 7. | Magnesium stearate | 3.0 | 1.00 |
| 8. | Strawberry flavor | 1.0 | 0.33 |

| | | |
|---|---|---|
| Total | 300.0 | 100 |

### Manufacturing Procedure:

1) Accurately weighed quantities of lamotrigine, spray dried mannitol, microcrystalline cellulose, aspartame, acesulfame, colloidal silicon dioxide and strawberry flavor were mixed together in a geometric order in a double cone blender.
2) The blend of step 1 was sifted through ASTM #40 mesh and uniformly mixed in a double cone blender for 12 minutes.
3) The weighed quantity of magnesium stearate was sifted through ASTM #40 mesh and added to blend of step 2 and then uniformly mixed in a double cone blender for 3 minutes.
4) The lubricated blend of step 3 was compressed using flat-faced beveled edge punches to form compressed tablets.

### Example 4

TABLE 5
Montelukast water dispersible tablet

| S. No | Ingredients | Quantity (mg/tab) | % w/w |
|---|---|---|---|
| 1. | Montelukast sodium | 5.20 | 1.85 |
| 2. | Spray dried mannitol | 103.0 | 36.79 |
| 3. | Microcrystalline cellulose | 161.30 | 57.61 |
| 4. | Aspartame | 2.0 | 0.71 |
| 5. | Acesulfame potassium | 1.0 | 0.36 |
| 6. | Colloidal silicon dioxide | 2.0 | 0.71 |
| 7. | Magnesium stearate | 4.50 | 1.61 |
| 8. | Strawberry flavor | 1.0 | 0.36 |

| | | |
|---|---|---|
| Total | 280 | 100 |

### Manufacturing Procedure:

1) Weighed quantities of spray-dried mannitol and microcrystalline cellulose were sifted through ASTM #40 mesh and uniformly mixed in a double cone blender.
2) The accurately weighed quantity of montelukast sodium was sifted through ASTM #40 mesh and mixed geometrically with the blend of step 1 in a double cone blender.
3) The weighed quantities of aspartame, acesulfame, colloidal silicon dioxide and strawberry flavor were sifted through ASTM #40 mesh and subsequently mixed with the blend of step 2 in a double cone blender for 12 minutes.
4) Weighed quantity of magnesium stearate was sifted through ASTM #60 mesh and uniformly mixed with the blend of step 3 in a double cone blender for 3 minutes.
3) The blend of step 2 was compressed using flat-faced beveled edge punches to form compressed tablets.

TABLE 6
Physicochemical properties of the tablet

| Parameter | Observed value |
|---|---|
| Hardness | 4-6 kp |
| Friability | 0.12% |
| Disintegration time | 27-31 sec |
| Dispersion quality test | No particles retained on 710 μm sieve |

### Example 5

TABLE 7
Olanzapine water dispersible tablet

| S. No | Ingredients | Quantity (mg/tab) | % w/w |
|---|---|---|---|
| 1. | Olanzapine | 5.00 | 1.79 |
| 2. | Spray dried mannitol | 103.0 | 36.79 |
| 3. | Microcrystalline cellulose | 161.50 | 57.68 |
| 4. | Aspartame | 2.0 | 0.71 |
| 5. | Acesulfame potassium | 1.0 | 0.36 |
| 6. | Colloidal silicon dioxide | 2.0 | 0.71 |
| 7. | Magnesium stearate | 4.50 | 1.61 |
| 8. | Strawberry flavor | 1.0 | 0.36 |

| | | |
|---|---|---|
| Total | 280 | 100 |

### Manufacturing Procedure:

1) Weighed quantities of spray-dried mannitol and microcrystalline cellulose were sifted through ASTM #40 mesh and uniformly mixed in a double cone blender.
2) The accurately weighed quantity of montelukast sodium was sifted through ASTM #40 mesh and mixed geometrically with the blend of step 1 in a double cone blender.
3) The weighed quantities of aspartame, acesulfame, colloidal silicon dioxide and strawberry flavor were sifted through ASTM #40 mesh and subsequently mixed together with the blend of step 2 in a double cone blender for 12 minutes.
4) Weighed quantity of magnesium stearate was sifted through ASTM #60 mesh and uniformly mixed with the blend of step 3 in a double cone blender for 3 minutes.
3) The blend of step 2 was compressed using flat-faced beveled edge punches to form compressed tablets.

TABLE 8
Physicochemical properties of the tablet

| Parameter | Observed value |
|---|---|
| Hardness | 3-5 kp |
| Friability | 0.25% |
| Disintegration time | 16-19 sec |
| Dispersion quality test | No particles retained on 710 μm sieve |

### Example-6

TABLE 9
Lamotrigine water dispersible tablet

| S. No | Ingredients | Quantity (mg/tab) | % w/w |
|---|---|---|---|
| 1. | Lamotrigine | 25.0 | 5.00 |
| 2. | Spray dried mannitol | 227.5 | 45.50 |
| 3. | Microcrystalline cellulose | 230.0 | 46.00 |
| 4. | Aspartame | 10 | 2.00 |
| 5. | Colloidal silicon dioxide | 3.0 | 0.60 |
| 6. | Magnesium stearate | 4.0 | 0.80 |
| 7. | Strawberry flavor | 0.5 | 0.10 |

| | | |
|---|---|---|
| Total | 500.0 | 100 |

### Manufacturing Procedure:

1) Accurately weighed quantities of lamotrigine, spray dried mannitol, microcrystalline cellulose, aspartame, colloidal silicon dioxide and strawberry flavor were sifted through ASTM #40 mesh.
2) The blend of step 1 was uniformly mixed in a double cone blender for 10 minutes.
3) The weighed quantity of magnesium stearate was added to the blend of step 2 and then uniformly mixed in a double cone blender for 3 minutes.
4) The blend of step 3 was compressed using flat-faced beveled edge punches to form compressed tablets.

TABLE 10
Physicochemical properties of the tablet

| Parameter | Observed value |
|---|---|
| Hardness | 8-11 kp |
| Friability | 0.13% |
| Disintegration time | 18-19 sec |
| Dispersion quality test | No particles retained on 710 μm sieve |

### Example 7

The tablets of example 3 of the present invention (Jubilant's water dispersible tablets) were evaluated as per the ICH guidelines. The tablets were stored in HDPE bottles at 40° C.±2° C. and 75% RH±5% RH for 3 months. The results of these studies are shown in the table 11 given below:

TABLE 11

| Parameters | Initial | 3 months |
|---|---|---|
| Average weight (mg) | 301 | 301 |
| Thickness (mm) | 3.58 | 3.58 |
| Hardness (kp) | 7-8 | 7-9 |
| Friability (%) | 0.1 | 0.08 |
| Dispersion disintegration | 17-21 sec | 20-21 sec |
| test (in water) | | |
| Dispersion quality test | No particles | No particles |
| | retained on | retained on |
| | 710 μm sieve | 710 μm sieve |
| Dissolution at 15 minutes in 900 | 96% | 90% |
| ml 0.1 N HCL (USP App. II @ 50 rpm) | | |
| Lamotrigine (% w/w) | 101.8 | 99.6 |
| Impurity A (% w/w) | ND* | ND* |
| Total related substances (% w/w) | ND* | ND* |

*Not Detected

It can be seen from the data given above, the dispersible tablets made in accordance with the present invention displayed excellent stability characteristics under accelerated stability conditions of 40° C.±2° C./75% RH±5% RH even after 3 months.

### Example 8

In order to assess physicochemical properties of the tablets of example 3 (Jubilant's water dispersible tablet), various tablet evaluation tests were performed. Physicochemical properties of the dispersible tablet of example 3 were compared with physicochemical properties of commercially available lamotrigine chewable dispersible tablet (Lamictal®CD) from Glaxosmithkline, USA. The results from the study were presented in the table below:

TABLE 12

| | Example 3 (Jubilant's | |
|---|---|---|
| | water dispersible | Lamictal ® |
| Parameters | lamotrigine tablet) | CD |
| Average weight (mg) | 300 | 64 |
| Thickness (mm) | 3.5 | 2.3 |
| Hardness (kp) | 7-8 | 3-4 |
| Friability (%) | 0.1 | 0.31 |
| Disintegration test | 17-21 sec | 34-35 sec |
| (in water) | | |
| Dispersion quality test | No particles | No particles |
| | retained on | retained on |
| | 710 μm sieve | 710 μm sieve |
| Dissolution at 15 minutes | 96 | 104 |
| in 900 ml, 0.1 N HCL | | |
| (USP App. II @ 50 rpm) | | |

From the above data it is clearly evident that composition of the present invention is an improvement over innovator's composition.

### Example 9

A comparative analysis was carried out in order to assess the stability of composition of example 3 (Jubilant's water dispersible tablet) vis-a-vis lamotrigine water dispersible tablets of example 3 of International Publication No WO2004/082587. The tablets of example 3 were subjected to accelerated stability testing at 40° C.±2° C. and 75% RH±5% RH for 3 months in HDPE bottles and observations were made initially and after 3 months. The results are summarized in table 13, given below:

TABLE 13

| | Example 3 (Jubilant's | | | |
|---|---|---|---|---|
| | water dispersible | | Example 3 of | |
| | lamotrigine tablet) | | WO2004/082587 | |
| Parameters | Initial | 3 months | Initial | 3 months |
| Lamotrigine (% w/w) | 101.8 | 99.6 | 99.67 | 99.43 |
| Impurity A (% w/w) | ND* | ND* | ND* | BLQ** |
| Total related | ND* | ND* | 0.043 | 0.054 |
| substances (% w/w) | | | | |

*Not Detected
**Below level of quantification

Although the foregoing invention has been described in some detail by way of illustrations and examples for the purposes of clarity of understanding, it will be obvious that certain changes and modifications may be practised within the scope of the appended claims. Modifications of the above-described modes of practising the invention that are obvious to person skilled in the art are intended to be included within the scope of the following claims.

1. A water dispersible compressed tablet comprising:
(a) about 0.01 to 50% w/w of lamotrigine or its pharmaceutically acceptable salts, hydrates or polymorphs;
(b) one or more water-soluble diluent;
(c) one or more water swellable diluent;
(d) optionally one or more pharmaceutically acceptable adjuvants; and
wherein the ratio of water-soluble diluent to water swellable diluent is from about 0.6 to about 0.9 and said tablet is essentially free of disintegrant, superdisintegrant and swellable clay.

2. The water dispersible tablet of claim 1, wherein said water soluble diluent is present in an amount of about 5% to about 50% by weight.

3. The water dispersible tablet of claim 1, wherein said water swellable diluent is present in an amount of about 35% to about 70% by weight.

4. The water dispersible compressed tablet of claim 1, wherein the pharmaceutically acceptable adjuvant is selected from one or more binders, lubricants, glidants, surfactants, sweeteners, flavors, stabilizers, or any combination thereof.

5. The water dispersible compressed tablet of claim 1, wherein the water soluble diluent is lactose, mannitol, xylitol, sorbitol, calcium sulfate dihydrate, inositol, dextrin, calcium sulfate anhydrous, fructose, kaolin, sugar compressible, sucrose, lactitol, dextrates, confectioner's sugar, sucrose, sodium chloride, dextrose, inorganic salts or any combination thereof.

6. The water dispersible compressed tablet of claim 5, wherein said water-soluble diluent is mannitol.

7. The water dispersible compressed tablet of claim 1, wherein the water swellable diluent is pregelatinized starch, starch, microcrystalline cellulose, powdered cellulose, silicified microcrystalline cellulose, dibasic calcium phosphate dihydrate, calcium phosphate, calcium carbonate, hydroxypropylcellulose, hydroxyethylcellulose, hydroxypropyl methylcellulose, polyethyleneglycol, xanthum gum, gum arabic or any combination thereof.

8. The water dispersible compressed tablet of claim 7, wherein said water swellable diluent is microcrystalline cellulose.

9. The water dispersible compressed tablet according to claim 1, wherein the water dispersible tablet is prepared by direct compression or wet granulation or dry granulation.

10. A process for manufacturing the water dispersible tablet according to claim 1, wherein the process comprises the steps of:
(a) forming a blend comprising lamotrigine or its pharmaceutically acceptable salts, hydrates or polymorph, one or more water soluble diluent, and one or more water swellable diluents and optionally one or more pharmaceutically acceptable adjuvant selected from binders, lubricants, glidants, surfactants, flavors, sweeteners, stabilizers or any combination thereof; and
(b) compressing the resultant blend into a tablet.

11. A process for manufacturing the water dispersible tablet according to claim 1, wherein the process comprises the steps of:
(a) forming a blend comprising lamotrigine or its pharmaceutically acceptable salts, hydrates or polymorph, and one or more water swellable diluents, and optionally one or more water soluble diluent and one or more pharmaceutically acceptable adjuvants selected from binders, lubricants, glidants, surfactants, stabilizers, flavors, sweeteners or any combinations thereof;
(b) granulating the blend with one or more binders to form granules;
(c) drying the resultant granules;
(d) blending the granules with water soluble diluents, optionally water swellable diluents and optionally one or more pharmaceutically acceptable adjuvants selected from lubricants, glidants, surfactants, sweeteners, flavors; and
(e) compressing the resultant blend into a tablet.

12. The water dispersible compound tablet of claims 5, wherein the inorganic salt is calcium carbonate.

13. The process of claim 11, wherein step (b) is performed by blending a solution or dispersion of one or more binders to form granules.

14. The process of claims 11, wherein step (b) is performed by adding one or more binders to the blend of step (a) and granulating the resulting blend with one or more suitable solvents.
